(19) United States
(12) Patent Application Publication
(10) Pub. No.: US 2009/0312238 A1
(43) Pub. Date: Dec. 17, 2009

(54) Protection Against Oxidative Damage in Cells

(75) Inventors: Daphna Sagher, Boca Raton, FL (US); David Brunell, West Palm Beach, FL (US); Herbert Weissbach, Boynton Beach, FL (US)

Correspondence Address: DARBY & DARBY P.C., P.O. BOX 770, Church Street Station, New York, NY, 10008-0770, US

(73) Assignee: Florida Atlantic University, Boca Raton, FL (US)

(21) Appl. No.: 12/299,109

(22) PCT Filed: May 3, 2007
(86) PCT No.: PCT/US07/10842

§ 371 Date: Feb. 4, 2009

Related U.S. Application Data
(60) Provisional application No. 60/797,249, filed on May 3, 2006.

(51) Int. Cl. A61K 38/02; C12N 5/06; G01N 33/53; A61P 39/06

(52) U.S. Cl. 514/6; 435/325; 435/375; 435/7.1

(57) ABSTRACT

The present invention relates to the use of MT as a reducing system for the Msr enzymes and other oxioreductase enzymes which form similar intermediates. Specifically, the invention provides for a reduction in the level of oxidative damage in cells through an increase in levels of MT by administration of suitable compounds, resulting in an increase in the activity of the Msr enzymes.

### STATEMENT OF FEDERALLY SPONSORED RESEARCH

The U.S. government may have certain rights to the invention by virtue of National Institutes of Health grant EY13022MK.

### FIELD OF INVENTION

The present invention relates, in general, to a method of reducing levels of oxidative damage in cells and, in particular, to a method of enhancing the level of activity of the methionine sulfoxide reductase family of enzymes and other oxioreductases involving similar intermediates through the induction of metallothionein or through other means of enhancing the reducing system of the enzymes. The invention also relates to compounds and compositions suitable for use in such methods.

### BACKGROUND

One of the primary mechanisms of aging in animals is considered to be oxidative stress, which is a normal but deleterious side effect of the metabolism required to sustain life. It is believed that a gradual accumulation of damage to cellular components caused by oxidative stress over time produces the aging pathology. Cells have natural mechanisms to limit oxidative stress or repair the damage caused by it. Improving the cell's ability to lower oxidative stress or enhance repair of oxidatively damaged components is believed to be an important technique for slowing the rate of aging and improving the health of older persons.

One type of oxidative damage to cells is the conversion of methionine to methionine sulfoxide. The methionine sulfoxide reductases (Msr) are a family of proteins that reduce methionine sulfoxide (Met(o)) back to methionine (Met) and protect cells against oxidative damage. The Msr system has also been shown to affect the life span of animals. The oxidation of Met to Met(o) results in the formation of two epimers of Met(o), called Met-S-(o) and Met-R-(o). MsrA (methionine sulfoxide reductase A) specifically reduces Met-S-(o), whether in peptide linkage or as a free amino acid. MsrB proteins specifically reduce the Met-R-(o) in proteins but have very weak activity with free Met-R-(o).

In animal cells, there is one gene that codes for MsrA but three separate genes that code for MsrB proteins. MsrA localizes primarily in the mitochondria or cytoplasm, whereas the three MsrB proteins have different subcellular localizations. MsrB1 (originally called Sel-x) is a selenoprotein primarily found in the cytoplasm and nucleus, MsrB2 (originally called CBS-1) is present mainly in the mitochondria and MsrB3 is localized primarily in the endoplasmic reticulum and mitochondria. The biological reducing system for MsrA appears to be reduced thioredoxin (Trx), and Trx has also been assumed to be the biological reductant for the MsrB proteins. However, detailed studies comparing the reducing system requirement for the MsrA and MsrB proteins have not been reported. Most in vitro studies have used DTT as the reducing agent for both MsrA and MsrB, because this agent appears to work very well with the Msr family of proteins that have been tested.

Trx is a small, 104-residue (in humans) oxioreductase that contains an active site at residues 31 through 34. This site contains two cysteines capable of forming a disulfide bond and is located on the surface of the protein where it may interact with other proteins. The formation of the disulfide bond is accompanied by a transfer of two electrons and two protons to the interacting protein which then becomes reduced.

Metallothionein (MT) is another protein which contains cysteine residues capable of forming intramolecular disulfide bonds. Historically, the primary function of metallothionein was believed to be related to the homeostasis of zinc and other metals. However, by virtue of the large number of cysteine residues and the small size of the protein, MT can also participate in oxidation-reduction reactions with a number of different substrates.

### SUMMARY

The present invention relates to the use of MT as a reducing system for the Msr enzymes and other oxioreductase enzymes which form similar intermediates. Specifically, the invention provides for a reduction in the level of oxidative damage in cells through an increase in levels of MT by administration of suitable compounds, resulting in an increase in the activity of the Msr enzymes.

In a preferred embodiment, a pharmaceutical composition comprises an isolated biological agent wherein the isolated biological agent is a thionein (T).

In another preferred embodiment, the thionein is a metallothionein, preferably a zinc metallothionein.

In another preferred embodiment, the pharmaceutical composition comprises zinc metallothionein and ethylenediaminetetraacetic acid (EDTA).

In another preferred embodiment, the composition comprises a reducing agent such as selenium and/or selenium derivatives. Examples of selenium derivatives include, but not limited to: selenocystamine, selenocysteamine, selenium dioxide; selenium sulfide; sodium selenite; sodium selenate; zinc selenite; copper selenate; barium selenite; ferrous selenide; hydrogen selenide; seleneous acid; selenic acid; sodium selenide; diphenyl selenide; benzeneseleninic anhydride; benzeneseleninic acid; diphenyl diselenide; selenophenol (phenylselenol); selenium aspartate; phenylselenenyl chloride; phenylselenenyl bromide; selenourea; L(+) selenomethionine; selenium tetrabromide.

In another preferred embodiment, a method of reducing, preventing or reversing oxidative damage in a cell, the method comprises the steps of: (a) providing a pharmaceutical composition comprising an isolated biological agent wherein the isolated biological agent is a thionein (T), the compound being a substrate for at least one Msr enzyme; (b) providing a cell expressing at least one Msr enzyme, said cell comprising or being exposed to reactive oxygen species; and, (c) contacting the cell with an amount of the compound sufficient to reduce, prevent, or reverse oxidative damage in the cell by said reactive oxygen species. Preferably, the cell is within an animal subject and the animal is suffering from a condition or disorder associated with oxidative damage.

In another preferred embodiment, a method of reducing, preventing or reversing oxidative damage in a cell, the method comprises the steps of: (a) providing a pharmaceutical composition comprising an isolated biological agent wherein the isolated biological agent is a thionein (T), the compound being a substrate for at least one Msr enzyme; (b) providing a cell expressing at least one Msr enzyme, said cell comprising or being exposed to reactive oxygen species; and, (c) contacting the cell with an amount of the compound sufficient to reduce, prevent, or reverse oxidative damage in the cell by said reactive oxygen species; wherein said method further comprises administering to said cell a pharmaceutical composition comprising sulindac, or sulindac metabolites, or sulindac derivatives or combinations thereof. Preferably, the composition further comprises selenium and selenium derivatives.

In another preferred embodiment, a method of treating a patient suffering from a condition or disorder associated with oxidative damage, the method comprises the steps of: (a) providing a pharmaceutical composition comprising an isolated biological agent wherein the isolated biological agent is a thionein (T), the compound being a substrate for at least one Msr enzyme; (b) administering to a patient the pharmaceutical composition; and, (c) contacting a cell with an amount of the compound sufficient to reduce, prevent, or reverse oxidative damage in the cell by said reactive oxygen species; thereby treating a patient suffering from a condition or disorder associated with oxidative damage.

In another preferred embodiment, a method of treating a patient suffering from a condition or disorder associated with oxidative damage, the method comprises the steps of: (a) providing a pharmaceutical composition comprising sulindac, an isolated biological agent wherein the isolated biological agent is a thionein (T), the compound being a substrate for at least one Msr enzyme, and/or a reducing agent; (b) administering to a patient the pharmaceutical composition; and, (c) contacting a cell with an amount of the compound sufficient to reduce, prevent, or reverse oxidative damage in the cell by said reactive oxygen species; thereby treating a patient suffering from a condition or disorder associated with oxidative damage. Examples of reducing agents include, but not limited to: ethylenediaminetetraacetic acid (EDTA); selenium and/or selenium derivatives. Examples of selenium derivatives include, but not limited to: selenocystamine, selenocysteamine, selenium dioxide; selenium sulfide; sodium selenite; sodium selenate; zinc selenite; copper selenate; barium selenite; ferrous selenide; hydrogen selenide; seleneous acid; selenic acid; sodium selenide; diphenyl selenide; benzeneseleninic anhydride; benzeneseleninic acid; diphenyl diselenide; selenophenol (phenylselenol); selenium aspartate; phenylselenenyl chloride; phenylselenenyl bromide; selenourea; L(+) selenomethionine; selenium tetrabromide.

In another preferred embodiment, a method of inducing metallothionein production in a cell comprises (a) providing a pharmaceutical composition comprising an isolated biological agent wherein the isolated biological agent is a thionein (T), the compound being a substrate for at least one Msr enzyme; (b) providing a cell expressing at least one Msr enzyme, said cell comprising or being exposed to reactive oxygen species; and (c) contacting the cell with an amount of the compound sufficient to induce metallothionein production. Preferably, the cell is within an animal subject or in vitro, such as in tissue culture. The method further comprises administering to said cell a pharmaceutical composition comprising sulindac, or sulindac metabolites, or sulindac derivatives or combinations thereof. Examples of selenium derivatives comprise at least one of: selenocystamine, selenocysteamine; selenium dioxide; selenium sulfide; sodium selenite; sodium selenate; zinc selenite; copper selenate; barium selenite; ferrous selenide; hydrogen selenide; seleneous acid; selenic acid; sodium selenide; diphenyl selenide; benzeneseleninic anhydride; benzeneseleninic acid; diphenyl diselenide; selenophenol (phenylselenol); selenium aspartate; phenylselenenyl chloride; phenylselenenyl bromide; selenourea; L(+) selenomethionine; and, selenium tetrabromide. The composition optionally comprises ethylenediaminetetraacetic acid (EDTA).

In another preferred embodiment, a method of diagnosing a patient suffering from a condition or disorder associated with oxidative damage comprises obtaining a biological sample from the patient; measuring metallothionein concentration in the sample; and, diagnosing a patient suffering from a condition or disorder associated with oxidative damage. Preferably, the patient is an animal. Examples of measuring the concentrations of metallothionein, Msr enzymes, oxidative damage, are described in detail in the examples which follow.

In one preferred embodiment, the biological sample is a cell, tissue, organ, blood or other fluids, such as for example, sputum, amniotic fluids, lymphatic fluids, vaginal fluids, and the like.

Other aspects of the invention are described infra.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIG. 1 is a graph showing the effect of heated liver S-100 concentration and EDTA on MsrB3 activity. Activity was measured without EDTA (∘-∘) or with 5 mM EDTA (-) in the reaction mix. MsrB3 (2 μg) was incubated with the indicated amount of heated S-100 in the absence of a reducing system (DTT or Trx), as described in Materials and Methods.

FIG. 2A is a graph showing the elution profile from a DE-52 column of the factor showing Msr activity and zinc content. Two peaks of reducing activity with hMsrB3 could be separated, and they are labeled MT-1 and MT-2. Activity (closed circles) is expressed as total nmoles DABS-Met formed per 1 ml fraction in the Msr reaction using hMsrB3. Zinc concentration (μM) is shown as open circles. FIG. 2 B is a graph showing the spectra of purified factor at pH 7.4 (-) and pH 2.0 (- - -). An extinction coefficient of ε₂₂₀=48,600 M⁻¹ cm⁻¹ at pH 2.0 was used to calculate the amount of MT in the fractions.

FIG. 3 is a graph showing T can supply the reducing system for hMsrB3 activity in the absence of EDTA. The incubations contained 4.5 μg of MsrB3, 20 nmoles of T or 20 nmoles of Zn-MT. The incubations with T did not contain EDTA, but 5 mM EDTA was added to the incubations with Zn-MT. At 20 minutes, Zn-MT in the absence of EDTA formed 1.3 nmoles, whereas T in the presence of 5 mM EDTA formed 23.5 nmoles. T (-), Zn-MT+EDTA (∘--∘).

FIG. 4 is a graph showing the reduction of T(o) by Trx. The preparation of T(o) and the incubation conditions are described in the text. The oxidation of NADPH was followed at 340 nm. Complete system (▪-▪), minus Trx (□-□), minus Trx reductase (∘-∘) and minus T(o) (-).

FIG. 5 is a schematic representation of the postulated role of Trx and MT in supplying the reducing requirement for the Msr enzymes.

FIGS. 6A and 6B show the structure of SeC (FIG. 6A) and selenite (FIG. 6B).

FIG. 7 is a graph showing the effect of selenocystamine concentration on the activity of hMsrB3 using either NADPH and Trx reductase (-) or T (∘-∘) as the primary reducing agent.

hMsrB3 (2.25 μg) was incubated at 37 degrees with the complete Trx system (Trx, Trx reductase and NADPH) or with 2 nmoles (T) as the reducing agent (see methods). Selenocystamine was added at the concentrations specified. Activity is expressed in nmoles Dabs-met(o) reduced by the enzyme in a 20-minute incubation.

FIG. 8 is a graph showing the time curve of hMsrB3 activity using the Trx reducing system either with (-) or without (∘-∘) 50 μM selenocystamine. Details of the reaction are as in FIG. 2. Only 1.0 nmole Dabs-L-met was formed in 20 minutes under the reaction conditions in the absence of selenocystamine.

FIGS. 9A and 9B are a schematic illustration showing a putative reaction mechanism showing reduction of Msr by selenocysteamine. There are two potential sources of hydrogen, either NADPH via Trx reductase (FIG. 9A) or thionein (FIG. 9B).

FIG. 10 is a graph showing selenocystamine (SeCm) can enhance MsrA activity in cardiomyocytes. Cardiomyocytes were incubated with 400 μM sulindac and indicated concentration of selenocystamine. Briefly, primary neonatal rat cardiomyocytes (approximately 10⁶ cells per treatment) were incubated with sulindac and two different concentrations of selenocystamine. Sulindac is a substrate for MsrA, and the amount of the reduced product, sulindac sulfide, indicates the amount of MsrA activity. The amount of product is 2.7, 3.7 and 5.6 picomoles for 0, 5 μM and 50 μM selenocystamine, respectively. This amounts to a 37% and 107% stimulation.

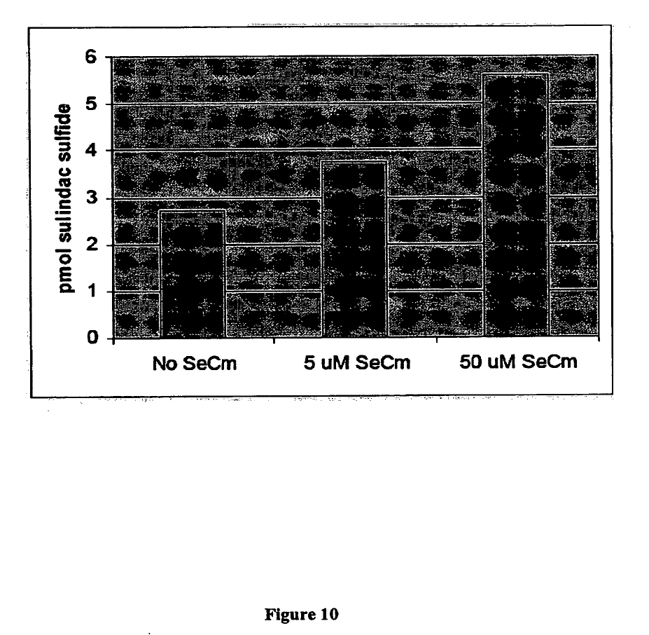

### DETAILED DESCRIPTION

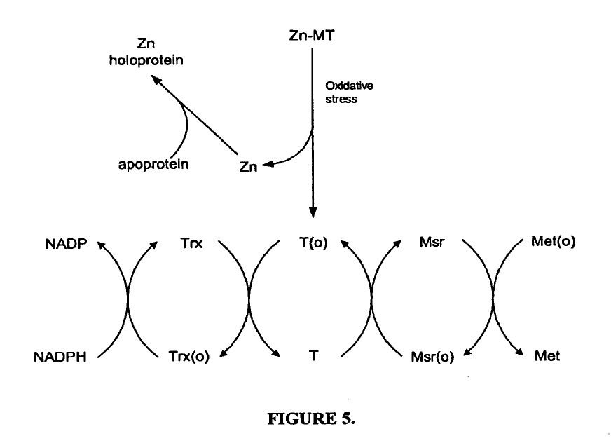

The present invention relates to methods of protecting cells against oxidative stress by the induction of metallothionein through the use of suitable compounds and the subsequent increase in MsrA and MsrB activity in cells. The reaction sequence is summarized in FIG. 5, in which cells, under oxidative stress, mobilize zinc from Zn-MT for use for the hundreds of zinc-containing proteins. The loss of the zinc from MT would also yield the cysteine-rich thionein. As shown in FIG. 5, thionein can subsequently reduce the oxidized Msr intermediates, either an enzyme-bound disulfide or sulfenic acid. This increase in the reducing power in the cell should stimulate Msr activity and protect cells against oxidative damage. Trx can reduce oxidized thionein and permit thionein to function as a cellular reducing agent and recycle as indicated. Trx may be only one of the possible cellular reducing systems that can reduce oxidized thionein. It is known that oxidized glutathione can oxidize MT and cause the release of Zn from Zn-MT and that reduced glutathione can reduce oxidized thionein, which can bind zinc.

### DEFINITIONS

In accordance with the present invention and as used herein, the following terms are defined with the following meanings, unless explicitly stated otherwise.

As used herein, “a”, “an,” and “the” include plural references unless the context clearly dictates otherwise.

As used herein, “disorders associated with oxidative stress” include any disease or disorder caused when cells are affected by oxidative stress. Examples, include, but not limited to: cancer, neurodegenerative disorders (e.g. Parkinson's Disease), atherosclerosis, mitochondrial damage, immune cell function and resultant disorders thereof.

As used herein, “cancer” refers to all types of cancer or neoplasm or malignant tumors found in mammals, including, but not limited to: leukemias, lymphomas, melanomas, carcinomas and sarcomas. Examples of cancers are cancer of the brain, breast, pancreas, cervix, colon, head & neck, kidney, lung, non-small cell lung, melanoma, mesothelioma, ovary, sarcoma, stomach, uterus and Medulloblastoma. The term “cancer” includes any cancer arising from a variety of chemical, physical, infectious organism cancer causing agents. For example, hepatitis B virus, hepatitis C virus, human papillomaviruses; sun; lead and lead compounds, X-rays, compounds found in grilled meats, and a host of substances used in textile dyes, paints and inks. Further details of cancer causing agents are listed in The Report on Carcinogens, Eleventh Edition. Federal law requires the Secretary of the Department of Health and Human Services to publish the report every two years.

“Diagnostic” or “diagnosed” means identifying the presence or nature of a pathologic condition or a patient susceptible to a disease. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. For example, an amount effective to delay the growth of or to cause a cancer, either a sarcoma or lymphoma, or to shrink the cancer or prevent metastasis. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

As used herein, a “pharmaceutical salt” include, but are not limited to, mineral or organic acid salts of basic residues such as amines; alkali or organic salts of acidic residues such as carboxylic acids. Preferably the salts are made using an organic or inorganic acid. These preferred acid salts are chlorides, bromides, sulfates, nitrates, phosphates, sulfonates, formates, tartrates, maleates, malates, citrates, benzoates, salicylates, ascorbates, and the like. The most preferred salt is the hydrochloride salt.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy.

The treatment of neoplastic disease, cancer, or neoplastic cells, refers to an amount of the composition, described throughout the specification and in the Examples which follow, capable of invoking one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention of tumor cell infiltration into peripheral organs; (6) inhibition, including (i) reduction, (ii) slowing down or (iii) complete prevention of metastasis; (7) enhancement of anti-tumor immune response, which may result in (i) maintaining tumor size, (ii) reducing tumor size, (iii) slowing the growth of a tumor, (iv) reducing, slowing or preventing invasion or (v) reducing, slowing or preventing metastasis; and/or (8) relief, to some extent, of one or more symptoms associated with the disorder.

The terms “dosing” and “treatment” as used herein refer to any process, action, application, therapy or the like, wherein a subject, particularly a human being, is rendered medical aid with the object of improving the subject's condition, either directly or indirectly.

The term “therapeutic compound” as used herein refers to a compound useful in the prophylaxis or treatment of disorders associated with oxidative damage and stress due to oxidative stress.

The term “therapeutic combination” as used herein refers to the administered therapeutic compounds when administered in combination therapy, and to any pharmaceutically acceptable carriers used to provide dosage forms such that the beneficial effect of each therapeutic compound is realized by the subject at the desired time, whether the compounds are administered substantially simultaneously, or sequentially.

“Biological samples” include solid and body fluid samples. The biological samples used in the present invention can include cells, protein or membrane extracts of cells, blood or biological fluids such as ascites fluid or brain fluid (e.g., cerebrospinal fluid). Examples of solid biological samples include, but are not limited to, samples taken from tissues of the central nervous system, bone, breast, kidney, cervix, endometrium, head/neck, gallbladder, parotid gland, prostate, pituitary gland, muscle, esophagus, stomach, small intestine, colon, liver, spleen, pancreas, thyroid, heart, lung, bladder, adipose, lymph node, uterus, ovary, adrenal gland, testes, tonsils and thymus. Examples of “body fluid samples” include, but are not limited to blood, serum, semen, prostate fluid, seminal fluid, urine, saliva, sputum, mucus, bone marrow, lymph, and tears.

“Sample” is used herein in its broadest sense. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

### Compositions

In a preferred embodiment, a pharmaceutical composition comprises an isolated biological agent wherein the isolated biological agent is a thionein (T).

In another preferred embodiment, the thionein is a metallothionein, preferably a zinc metallothionein.

In another preferred embodiment, the pharmaceutical composition comprises zinc metallothionein and ethylenediaminetetraacetic acid (EDTA).

In another preferred embodiment, the composition comprises a reducing agent such as for example selenium and/or selenium derivatives. Examples of selenium derivatives include, but not limited to: selenocystamine, selenocysteamine, selenium dioxide; selenium sulfide; sodium selenite; sodium selenate; zinc selenite; copper selenate; barium selenite; ferrous selenide; hydrogen selenide; seleneous acid; selenic acid; sodium selenide; diphenyl selenide; benzeneseleninic anhydride; benzeneseleninic acid; diphenyl diselenide; selenophenol (phenylselenol); selenium aspartate; phenylselenenyl chloride; phenylselenenyl bromide; selenourea; L(+) selenomethionine; selenium tetrabromide.

In another preferred embodiment, the composition comprises sulindac. The sulindac ranges from 1% to 70% w/w of sulindac. Other preferred amounts of sulindac include (in w/w) 10% sulindac, 15% sulindac, 20% sulindac, 50% sulindac. The compounds used in the treatment of this invention are effective on precancerous and cancerous lesions either because they are active themselves or because they are metabolized to active derivatives. When administered as a combination, the therapeutic agents can be formulated as separate compositions which are given at the same time or different times, or the therapeutic agents can be given as a single composition. The two components may be applied sequentially. In such a sequential application, the sulindac will have to be applied first followed by the peroxide. Preferably, the applications are made within one hour and most preferably, they are made within about one half hour of each other.

### Animal Subjects

Because oxidative damage to cells is a ubiquitous phenomenon, the invention is believed to be compatible with any animal subject. A non-exhaustive list of examples of such animals includes mammals such as mice, rats, rabbits, goats, sheep, pigs, horses, cattle, dogs, cats, and primates such as monkeys, apes, and human beings. Those animal subjects that have a disease or condition that relates to oxidative damage are preferred for use in the invention as these animals may have the symptoms of their disease reduced or even reversed. In particular, human patients suffering from inflammation, chronic obstructive lung diseases such as emphysema, reperfusion damage after heart attack or stroke, neurodegenerative diseases (for example, Parkinson's disease, Alzheimer's disease, and ALS), autoimmune diseases such as rheumatoid arthritis, lupus, and Crohn's disease, conditions related to premature birth, conditions caused by exposure to ultraviolet light, and age-related conditions (as but one example, age-related degenerative conditions of the eye including age-related macular degeneration and cataract formation) are suitable animal subjects for use in the invention. In the experiments described herein, animals used for demonstration of beneficial effects of protection against ROS damage by the compounds of the invention are the fruit fly and the mouse. Nonetheless, by adapting the methods taught herein to other methods known in medicine or veterinary science (for example, adjusting doses of administered substances according to the weight of the subject animal), the compounds and compositions of the invention can be readily optimized for use in other animals.

### Administration of Compositions

In another preferred embodiment, a method of reducing, preventing or reversing oxidative damage in a cell, the method comprises the steps of: (a) providing a pharmaceutical composition comprising an isolated biological agent wherein the isolated biological agent is a thionein (T), the compound being a substrate for at least one Msr enzyme; (b) providing a cell expressing at least one Msr enzyme, said cell comprising or being exposed to reactive oxygen species; and, (c) contacting the cell with an amount of the compound sufficient to reduce, prevent, or reverse oxidative damage in the cell by said reactive oxygen species. Preferably, the cell is within an animal subject and the animal is suffering from a condition or disorder associated with oxidative damage.

In another preferred embodiment, a method of reducing, preventing or reversing oxidative damage in a cell, the method comprises the steps of: (a) providing a pharmaceutical composition comprising an isolated biological agent wherein the isolated biological agent is a thionein (T), the compound being a substrate for at least one Msr enzyme; (b) providing a cell expressing at least one Msr enzyme, said cell comprising or being exposed to reactive oxygen species; and, (c) contacting the cell with an amount of the compound sufficient to reduce, prevent, or reverse oxidative damage in the cell by said reactive oxygen species; wherein said method further comprises administering to said cell a pharmaceutical composition comprising sulindac, or sulindac metabolites, or sulindac derivatives or combinations thereof. Preferably, the composition further comprises selenium and selenium derivatives.

In another preferred embodiment, a method of treating a patient suffering from a condition or disorder associated with oxidative damage, the method comprises the steps of: (a) providing a pharmaceutical composition comprising sulindac, an isolated biological agent wherein the isolated biological agent is a thionein (T), the compound being a substrate for at least one Msr enzyme, and/or a reducing agent; (b) administering to a patient the pharmaceutical composition; and, (c) contacting a cell with an amount of the compound sufficient to reduce, prevent, or reverse oxidative damage in the cell by said reactive oxygen species; thereby treating a patient suffering from a condition or disorder associated with oxidative damage. Examples of reducing agents include, but not limited to: ethylenediaminetetraacetic acid (EDTA); selenium and/or selenium derivatives. Examples of selenium derivatives include, but not limited to: selenocystamine, selenocysteamine, selenium dioxide; selenium sulfide; sodium selenite; sodium selenate; zinc selenite; copper selenate; barium selenite; ferrous selenide; hydrogen selenide; seleneous acid; selenic acid; sodium selenide; diphenyl selenide; benzeneseleninic anhydride; benzeneseleninic acid; diphenyl diselenide; selenophenol (phenylselenol); selenium aspartate; phenylselenenyl chloride; phenylselenenyl bromide; selenourea; L(+) selenomethionine; selenium tetrabromide.

In another preferred embodiment, a method of inducing metallothionein production in a cell comprises (a) providing a pharmaceutical composition comprising an isolated biological agent wherein the isolated biological agent is a thionein (T), the compound being a substrate for at least one Msr enzyme; (b) providing a cell expressing at least one Msr enzyme, said cell comprising or being exposed to reactive oxygen species; and (c) contacting the cell with an amount of the compound sufficient to induce metallothionein production. Preferably, the cell is within an animal subject or in vitro, such as in tissue culture. The method further comprises administering to said cell a pharmaceutical composition comprising sulindac, or sulindac metabolites, or sulindac derivatives or combinations thereof. Examples of selenium derivatives comprise at least one of: selenocystamine, selenocysteamine; selenium dioxide; selenium sulfide; sodium selenite; sodium selenate; zinc selenite; copper selenate; barium selenite; ferrous selenide; hydrogen selenide; seleneous acid; selenic acid; sodium selenide; diphenyl selenide; benzeneseleninic anhydride; benzeneseleninic acid; diphenyl diselenide; selenophenol (phenylselenol); selenium aspartate; phenylselenenyl chloride; phenylselenenyl bromide; selenourea; L(+) selenomethionine; and, selenium tetrabromide. The composition optionally comprises ethylenediaminetetraacetic acid (EDTA).

In another preferred embodiment, a method of diagnosing a patient suffering from a condition or disorder associated with oxidative damage comprises obtaining a biological sample from the patient; measuring metallothionein concentration in the sample; and, diagnosing a patient suffering from a condition or disorder associated with oxidative damage. Preferably, the patient is an animal.

In one preferred embodiment, the biological sample is a cell, tissue, organ, blood or other fluids, such as for example, sputum, amniotic fluids, lymphatic fluids, vaginal fluids, and the like.

The pharmaceutical compositions of the invention may be administered to animals including humans in any suitable formulation. For example, the compositions may be formulated in pharmaceutically acceptable carriers or diluents such as physiological saline or a buffered salt solution. Suitable carriers and diluents can be selected on the basis of mode and route of administration and standard pharmaceutical practice. A description of other exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions, or enhance the activity of the Msr system. One such enhancing substance could be nicotinamide which is part of the molecule, NADPH, that supplies the reducing power to the reaction catalyzed by the members of the Msr family.

In a preferred embodiment the compositions can include sulindac or derivatives thereof. Sulindac can be administered as part of the pharmaceutical composition and/or be administered on its own either before, during and/or after treatment with the compositions of the invention. Sulindac has been particularly well received among the NSAIDs for gastrointestinal polyp treatment. Sulindac is a sulfoxide compound that itself is believed to be inactive as an anti-arthritic agent. The sulfoxide is known to be converted by liver and other tissues to the corresponding sulfide, which is acknowledged to be the active moiety as a prostaglandin inhibitor. Recently, this conversion has been shown to be catalyzed by methionine sulfoxide reductase (MsrA). The sulfide, however, is associated with the side effects of conventional NSAIDs. Sulindac appears to be metabolized to sulindac sulfone by as yet unknown reactions. Sulindac sulfone is not an inhibitor of prostaglandin synthesis but has apoptotic activity against a wide array of cancer cells. The sulfone is currently being evaluated in Phase 2-3 clinical trials as therapy for multiple different types of cancers.

The compositions of the invention may be administered to animals by any conventional technique. Such administration may be oral or parenteral (for example, by intravenous, subcutaneous, intramuscular, or intraperitoneal introduction). The compositions may also be administered directly to the target site by, for example, surgical delivery to an internal or external target site, or by catheter to a site accessible by a blood vessel. Other methods of delivery, for example, liposomal delivery or diffusion from a device impregnated with the composition, are known in the art. The compositions may be administered in a single bolus, multiple injections, or by continuous infusion (for example, intravenously or by peritoneal dialysis). For parenteral administration, the compositions are preferably formulated in a sterilized pyrogen-free form.

Compositions of the invention can also be administered in vitro to a cell (for example, to prevent oxidative damage during ex vivo cell manipulation, for example of organs used for organ transplantation or in in vitro assays) by simply adding the composition to the fluid in which the cell is contained.

### Effective Doses

An effective amount is an amount which is capable of producing a desirable result in a treated animal or cell (for example, reduced oxidative damage to cells in the animal or cell). As is well known in the medical and veterinary arts, dosage for any one animal depends on many factors, including the particular animal's size, body surface area, age, the particular composition to be administered, time and route of administration, general health, and other drugs being administered concurrently. It is expected that an appropriate dosage for parenteral or oral administration of compositions of the invention would be in the range of about 1 μg to 100 mg/kg of body weight in humans. An effective amount for use with a cell in culture will also vary, but can be readily determined empirically (for example, by adding varying concentrations to the cell and selecting the concentration that best produces the desired result). It is expected that an appropriate concentration would be in the range of about 0.0001-100 mM. More specific dosages can be determined by the method described below.

Toxicity and efficacy of the compositions of the invention can be determined by standard pharmaceutical procedures, using cells in culture and/or experimental animals to determine the LD₅₀ (the dose lethal to 50% of the population) and the ED₅₀ (the dose that effects the desired result in 50% of the population). Compositions that exhibit a large LD₅₀/ED₅₀ ratio are preferred. Although less toxic compositions are generally preferred, more toxic compositions may sometimes be used in in vivo applications if appropriate steps are taken to minimize the toxic side effects.

Data obtained from cell culture and animal studies can be used in estimating an appropriate dose range for use in humans. A preferred dosage range is one that results in circulating concentrations of the composition that cause little or no toxicity. The dosage may vary within this range depending on the form of the composition employed and the method of administration.

### Formulations

A compound of the present invention can be formulated as a pharmaceutical composition. Such a composition can then be administered orally, parenterally, by inhalation spray, rectally, or topically in dosage unit formulations containing conventional nontoxic pharmaceutically acceptable carriers, adjuvants, and vehicles as desired. Topical administration can also involve the use of transdermal administration such as transdermal patches or iontophoresis devices. The term parenteral as used herein includes subcutaneous injections, intravenous, intramuscular, intrasternal injection, inhalation or infusion techniques.

Suppositories for rectal administration of the drug can be prepared by mixing the drug with a suitable nonirritating excipient such as cocoa butter, synthetic mono- di- or triglycerides, fatty acids and polyethylene glycols that are sold at ordinary temperatures but liquid at the rectal temperature and will therefore melt in the rectum and release the drug.

The methods and combinations of the present invention provide one or more benefits. Combinations of the present invention may allow for a lower dose of each agent. A benefit of lowering the dose of the compounds, compositions, agents and therapies of the present invention administered to a mammal includes a decrease in the incidence of adverse effects associated with higher dosages.

By lowering the incidence of adverse effects, an improvement in the quality of life of a patient undergoing treatment for cancer is contemplated. Further benefits of lowering the incidence of adverse effects include an improvement in patient compliance, a reduction in the number of clinical visits needed for the treatment of adverse effects, and a reduction in the administration of analgesic agents needed to treat pain associated with the adverse effects.

Alternatively, the methods and combination of the present invention can also maximize the therapeutic effect at higher doses.

Formulations suitable for topical administration include liquid or semi-liquid preparations suitable for penetration through the skin to the site of where treatment is required, such as liniments, lotions, creams, ointments or pastes, and drops suitable for administration to the eye, ear, or nose. Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified and sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

The composition of the invention can be administered to a patient either by themselves, or in pharmaceutical compositions where it is mixed with suitable carriers or excipient(s). In treating a patient exhibiting a disorder of interest, a therapeutically effective amount of a agent or agents such as these is administered. A therapeutically effective dose refers to that amount of the compound that results in amelioration of symptoms or a prolongation of survival in a patient.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized.

For any compound used in the method of the invention, the therapeutically effective dose can be estimated initially from cell culture assays. For example, a dose can be formulated in animal models to achieve a circulating plasma concentration range that includes the IC₅₀ as determined in cell culture. Such information can be used to more accurately determine useful doses in humans. Levels in plasma may be measured, for example, by HPLC.

The exact formulation, route of administration and dosage can be chosen by the individual physician in view of the patient's condition. (See e.g. Fingl et al., in The Pharmacological Basis of Therapeutics, 1975, Ch. 1 p. 1). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administrated dose in the management of the oncogenic disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Depending on the specific conditions being treated, such agents may be formulated and administered systemically or locally. Techniques for formulation and administration may be found in Remington's Pharmaceutical Sciences, 18^{th} ed., Mack Publishing Co., Easton, Pa. (1990). Suitable routes may include oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections, just to name a few.

The compositions described above may be administered to a subject in any suitable formulation. In addition to treatment of cancer with topical formulations of the composition, in other aspects of the invention the composition can be delivered by other methods. For example, the composition can be formulated for parenteral delivery, e.g., for subcutaneous, intravenous, intramuscular, or intratumoral injection. Other methods of delivery, for example, liposomal delivery or diffusion from a device impregnated with the composition might be used. The compositions may be administered in a single bolus, multiple injections, or by continuous infusion (for example, intravenously or by peritoneal dialysis). For parenteral administration, the compositions are preferably formulated in a sterilized pyrogen-free form. Compositions of the invention can also be administered in vitro to a cell (for example, to induce apoptosis in a cancer cell in an in vitro culture) by simply adding the composition to the fluid in which the cell is contained.

Depending on the specific conditions being treated, such agents may be formulated and administered systemically or locally. Techniques for formulation and administration may be found in Remington's Pharmaceutical Sciences, 18^{th} ed., Mack Publishing Co., Easton, Pa. (1990). Suitable routes may include oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections, just to name a few.

For injection, the agents of the invention may be formulated in aqueous solutions, preferably in physiologically compatible buffers such as Hanks's solution, Ringer's solution, or physiological saline buffer. For such transmucosal administration, penetrants appropriate to the barrier to be permeated are used in the formulation. Such penetrants are generally known in the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to their hydrophobicity, small organic molecules may be directly administered intracellularly.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Formulations suitable for topical administration include liquid or semi-liquid preparations suitable for penetration through the skin to the site of where treatment is required, such as liniments, lotions, creams, ointments or pastes, and drops suitable for administration to the eye, ear, or nose. Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified and sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogels. The formulation may incorporate any suitable surface active agent such as an anionic, cationic or non-ionic surface active such as sorbitan esters or polyoxyethylene derivatives thereof. Suspending agents such as natural gums, cellulose derivatives or inorganic materials such as silicaceous silicas, and other ingredients such as lanolin, may also be included.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxy-methylcellulose, and/or polyvinyl pyrrolidone (PVP). If desired, disintegrating agents may be added, such as the cross-linked polyvinyl pyrrolidone, agar, or alginic acid or a salt thereof such as sodium alginate.

Dragee cores are provided with suitable coating. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

The composition can include a buffer system, if desired. Buffer systems are chosen to maintain or buffer the pH of compositions within a desired range. The term “buffer system” or “buffer” as used herein refers to a solute agent or agents which, when in a water solution, stabilize such solution against a major change in pH (or hydrogen ion concentration or activity) when acids or bases are added thereto. Solute agent or agents which are thus responsible for a resistance or change in pH from a starting buffered pH value in the range indicated above are well known. While there are countless suitable buffers, potassium phosphate monohydrate is a preferred buffer.

The final pH value of the pharmaceutical composition may vary within the physiological compatible range. Necessarily, the final pH value is one not irritating to human skin and preferably such that transdermal transport of the active compound, i.e. sulindac, peroxide, arsenic trioxide is facilitated. Without violating this constraint, the pH may be selected to improve the compound stability and to adjust consistency when required. In one embodiment, the preferred pH value is about 3.0 to about 7.4, more preferably about 3.0 to about 6.5, most preferably from about 3.5 to about 6.0.

For preferred topical delivery vehicles the remaining component of the composition is water, which is necessarily purified, e.g., deionized water. Such delivery vehicle compositions contain water in the range of more than about 50 to about 95 percent, based on the total weight of the composition. The specific amount of water present is not critical, however, being adjustable to obtain the desired viscosity (usually about 50 cps to about 10,000 cps) and/or concentration of the other components. The topical delivery vehicle preferably has a viscosity of at least about 30 centipoises.

Other known transdermal skin penetration enhancers can also be used to facilitate delivery of the composition. Illustrative are sulfoxides such as dimethylsulfoxide (DMSO) and the like; cyclic amides such as 1-dodecylazacycloheptane-2-one (AZONE™, a registered trademark of Nelson Research, Inc.) and the like; amides such as N,N-dimethyl acetamide (DMA) N,N-diethyl toluamide, N,N-dimethyl formamide, N,N-dimethyl octamide, N,N-dimethyl decamide, and the like; pyrrolidone derivatives such as N-methyl-2-pyrrolidone, 2-pyrrolidone, 2-pyrrolidone-5-carboxylic acid, N-(2-hydroxyethyl)-2-pyrrolidone or fatty acid esters thereof, 1-lauryl-4-methoxycarbonyl-2-pyrrolidone, N-tallowalkylpyrrolidones, and the like; polyols such as propylene glycol, ethylene glycol, polyethylene glycol, dipropylene glycol, glycerol, hexanetriol, and the like; linear and branched fatty acids such as oleic, linoleic, lauric, valeric, heptanoic, caproic, myristic, isovaleric, neopentanoic, trimethyl hexanoic, isostearic, and the like; alcohols such as ethanol, propanol, butanol, octanol, oleyl, stearyl, linoleyl, and the like; anionic surfactants such as sodium laurate, sodium lauryl sulfate, and the like; cationic surfactants such as benzalkonium chloride, dodecyltrimethylammonium chloride, cetyltrimethylammonium bromide, and the like; non-ionic surfactants such as the propoxylated polyoxyethylene ethers, e.g., Poloxamer 231, Poloxamer 182, Poloxamer 184, and the like, the ethoxylated fatty acids, e.g., Tween 20, Myrj 45, and the like, the sorbitan derivatives, e.g., Tween 40, Tween 60, Tween 80, Span 60, and the like, the ethoxylated alcohols, e.g., polyoxyethylene (4) lauryl ether (Brij 30), polyoxyethylene (2) oleyl ether (Brij 93), and the like, lecithin and lecithin derivatives, and the like; the terpenes such as D-limonene, α-pinene, β-carene, α-terpineol, carvol, carvone, menthone, limonene oxide, α-pinene oxide, eucalyptus oil, and the like. Also suitable as skin penetration enhancers are organic acids and esters such as salicyclic acid, methyl salicylate, citric acid, succinic acid, and the like.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification.

All publications and patent documents cited in this application are incorporated by reference in pertinent part for all purposes to the same extent as if each individual publication or patent document were so individually denoted. By their citation of various references in this document, Applicants do not admit any particular reference is “prior art” to their invention.

### EXAMPLES

### Materials and Methods

Methionine sulfoxide, Dabsyl chloride (4-N,N-dimethylaminoazobenzene-4-sulfonyl chloride, DABS-Cl), PAR (4-(2-pyridylazo)resorcinol) and other chemicals including rabbit liver Zn-MT were purchased from Sigma-Aldrich, unless specified otherwise. DABS-met-S-(o) and DABS-met-R-(o) were prepared by derivatizing the amino group of the met-R-(o) or met-S-(o) epimers with DABS-Cl (Lavine, T. (1947) J. Biol. Chem. 169, 477-491; Minetti, G., Balduini, C. & Brovelli, A. (1994) Ital J Biochem 43, 273-283). Trx and Trx reductase (E. coli) were overexpressed and purified from E. coli, and human Trx was purchased from Sigma. Rat Trx reductase (TR3) was a generous gift from Vadim Gladyshev, University of Nebraska. Clones for bovine MsrA (bMsrA), eMsrA, eMsrB and hMsrB2, were overexpressed in E. coli, and the respective proteins were purified as described elsewhere (Lowther, W. T., Weissbach, H., Etienne, F., Brot, N. & Matthews, B. W. (2002) Nat Struct Biol 9, 348-352; Rahman, M. A., Nelson, H., Weissbach, H. & Brot, N. (1992) J Biol Chem 267, 15549-15551; Moskovitz, J., Weissbach, H. & Brot, N. (1996) Proc Natl Acad Sci USA 93, 2095-2099). The hMsrB3 cDNA from human lens was amplified by PCR, cloned into a pET vector and overexpressed in BL21 E. coli cells. The harvested cells were suspended in 1/100 volume of original culture using 50 mM Tris pH 7.4. After sonication and centrifugation at 10,000×g, the supernatant was fractionated on a Sephadex G-75 column. Active fractions were combined, and protein purity (>80%) was confirmed by SDS-PAGE.

Purification of an Active Factor from Bovine Liver.

Fresh bovine liver was homogenized in 3 volumes of 50 mM Tris, pH 7.4, centrifuged at 10,000×g for 30 minutes and then at 100,000×g for 16 hours (S-100). The S-100 fraction was heated at 80° C. for 5 minutes and centrifuged to remove precipitated proteins (heated S-100). Once the active material was suspected to be a MT, further purification followed an established method for MT (Vasak, M. (1991) Methods Enzymol 205, 41-44). Using a Bio-Rad DuoFlow HPLC system, the heated S-100 was placed on a sizing column (Superdex 75 HR 10/30) followed by DE-52 anion-exchange chromatography. The fractions were routinely monitored at 240 and 280 nm. Two distinct peaks of activity were eluted from the DE-52 column that corresponded to Zn-MT-1 and Zn-MT-2, as described in the results.

### Preparation of Thionein (1) and Oxidized Thionein (T(o)) and Assay of T(o) Reduction by Trx.

T and T(o) were prepared from Zn-MT by modification of a previously-described procedure (Klein, D., Sato, S. & Summer, K. H. (1994) Anal Biochem 221, 405-409). Briefly, purified Zn-MT was dialyzed against 10 mM HCl (pH 2.0) containing 150 mM NaCl for 12 hours at 4° C. The protein after dialysis is reduced, metal-free T and appears stable when left at pH 2.0. To study the activity of T in the Msr system, T was neutralized and added to the reaction mixtures immediately before the incubations were initiated. To oxidize T, 0.75 volumes of 50 mM Tris base were added to the T sample to bring the pH to 8.5. Under these conditions, approximately 50% of the sulfhydryls will become oxidized after 4 hours at room temperature or 2 hours at 37° C. With longer incubations, the T(o) started to precipitate. The assay for free sulfhydryl groups used DTNB as described elsewhere (Li, T. Y., Minkel, D. T., Shaw, C. F. 3. & Petering, D. H. (1981) Biochem J 193, 441-446).

To study the reduction of T(o) by Trx, the reaction mixtures contained, in a total volume of 1 ml, 100 μM NADPH, 26 μg Trx, 6 μg TrxB and 28 μg of partially-oxidized T (see above). The oxidation of NADPH was followed at 340 nm at room temperature.

### Analysis of Zinc Content.

Zinc was quantitatively determined in the MT preparations using the PAR reagent (Hunt, J. B., Neece, S. H. & Ginsburg, A. (1985) Anal Biochem 146, 150-157; Shaw, C. F. III, Savas, M. M. & Petering, D. H. (1991) Methods Enzymol 205, 401-414). The samples (100 μl) were incubated with 10 mM NEM for 1 hour at room temperature. PAR (100 nmoles) was then added and the sample diluted with water to 1 ml. The Zn-PAR complex was measured at 500 nm. 10 nmoles of zinc gave a reading of 0.720 at 500 nm. A complete metals analysis of the purified MT preparation was performed by Dr. Joseph Caruso, University of Cincinnati, using an Agilent 7500 ICP-MS. Molecular weight determinations of the purified protein were performed by Dr. Peter Yau, University of Illinois, using electrospray mass spectrometry.

### Colorimetric Assay for Msr Activity Based on the Reduction of Dabs-Met(o).

The reaction mixture (200 μl) to measure Msr activity contained 100 mM Tris-Cl pH 7.4, 100 nmoles of the indicated DABS-met(o) epimer (see Materials), either 15 mM DTT or the Trx regenerating system (Trx, 10 μg, Trx reductase, 2.4 μg, NADPH, 500 μM) and Msr enzyme as indicated. When the liver fractions (Zn-MT, T or T(o)) were tested, DTT was omitted and the Trx reducing system and 5 mM EDTA were added as indicated. Incubations were for 60 minutes at 37° C. unless noted otherwise. In experiments using T(o), the incubations did not contain EDTA. The quantitation of DABS-met formed employed a slight modification of an extraction procedure previously described by Etienne et al (Etienne, F., Resnick, L., Sagher, D., Brot, N. & Weissbach, H. (2003) Biochem Biophys Res Commun 312, 1005-1010) for the reduction of sulindac to sulindac sulfide by MsrA. The reactions were stopped by the addition of 200 μl of 1 M sodium acetate pH 6.0, followed by the addition of 100 μL of acetonitrile and 1 ml of benzene. After thorough shaking and centrifugation, the optical density of the benzene layer was read at 436 nm. 100 nmoles of the product, DABS-L-met, carried through this procedure, gave an optical density reading of 1.7, whereas 100 nmoles of the substrate, either the R or the S epimer of DABS-met(o), read less than 0.04. Under these conditions, the reaction was proportional to Msr concentration until more than 75% of the substrate was reduced. Unless indicated otherwise, the results are presented as nmoles DABS-met formed in 60 minutes. As little as two nmoles of product could be measured. This assay could be used with purified preparations of MsrA and MsrB as well as crude extracts of mammalian tissues. Bacterial extracts, in the presence of a reducing system, destroyed the substrate, and further studies are needed to adapt the assay to bacterial extracts.

### Example 1

### Reduced Trx is not an Efficient Reducing Agent for Human MsrB2 and MsrB3

In the course of developing the DABS colorimetric assay for Msr activity (see Methods), it was confirmed that eMsrA and eMsrB could use either DTT or Trx to supply the reducing power, with similar or more activity observed with Trx, in vitro. However, although both hMsrB2 and hMsrB3 could use DTT as the reducing agent, these proteins showed very little activity with Trx. Table 1 compares the activity of several recombinant Msr proteins using either DTT or Trx. It can be seen that eMsrA, bMsrA and eMsrB are active with either DTT or Trx as the reducing system. In fact, eMsrB was much more active with Trx than with DTT. In contrast, hMsrB2 and hMsrB3 work very poorly with Trx, having less than 10% of the activity seen with DTT. One possibility was that the human MsrB proteins specifically required mammalian Trx and not the bacterial Trx that was used in these experiments. We therefore tested hMsrB3, as well as eMsrA and eMsrB, with mammalian Trx and mammalian Trx reductase. Reduced mammalian Trx, like the bacterial Trx, gave very poor activity with hMsrB3, but both eMsrA and eMsrB efficiently used Trx from either source. The weak activity of hMsrB2 and hMsrB3 with Trx suggested that there may be another reducing system for these proteins in mammalian cells that either functions in place of Trx or is an intermediate hydrogen carrier between Trx and the human MsrB proteins.

TABLE 1
Thioredoxin and DTT as reducing agents with various Msr proteins

| | nmoles DABS-met | | |
|---|---|---|---|
| Msr Proteins | Trx | DTT | Ratio Trx/DTT |
| eMsrA | 46.8 | 46.2 | 1.0 |
| eMsrB | 60.3 | 11.1 | 5.4 |
| bMsrA | 15.8 | 30.0 | 0.53 |
| hMsrB2 | 0.8 | 31.3 | 0.03 |
| hMsrB3 | 1.7 | 53.8 | 0.03 |

DABS-met-S-(o) was used as substrate with MsrA proteins and DABS-met-R-(o), with MsrB proteins (e-E. coli, b-bovine, h-human). The incubation conditions and assay are described in Methods. The amounts of Msr protein used were: eMsrA, 1.6 μg; eMsrB, 2.7 μg; bMsrA, 2 μg; hMsrB2, 2.7 μg; hMsrB3, 2.3 μg.

### Example 2

### Zn-MT in the Presence of EDTA can Serve as a Reducing Agent for Msr

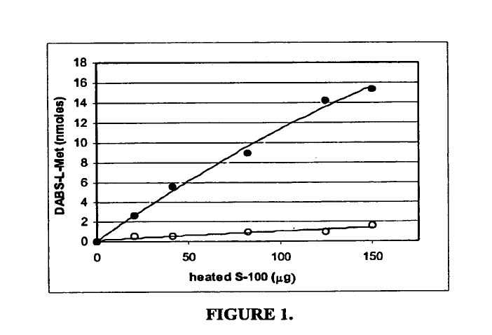

In our attempts to search for a biological factor that was more efficient than Trx in supplying the reducing system for hMsrB2 and hMsrB3, we initially tested an S-100 from bovine liver. Using hMsrB3, we were able to detect significant reducing activity in the liver S-100 fraction, but only in the presence of EDTA. The active material was stable to heating at 80° C. for 10 minutes. FIG. 1 shows the effect of protein concentration of the heated S-100 extract and the almost complete dependency on EDTA for hMsrB3 activity. Optimal activity was seen with levels of EDTA above 2.5 mM. Routinely, 5 mM EDTA has been used in the experiments. EDTA by itself had no significant effect on hMsrB3 activity, although hMsrB3 contains zinc. Other chelating agents were tested in place of EDTA with Zn-MT. 1,10-phenanthroline (5 mM) gave about 40% of the activity of EDTA, whereas EGTA (5 or 20 mM), deferoxamine (5 mM) and zincon (500 μM) were inactive. Thus, EDTA was used throughout the present studies. A series of metal salts could not replace EDTA or the heated S-100 in the reaction.

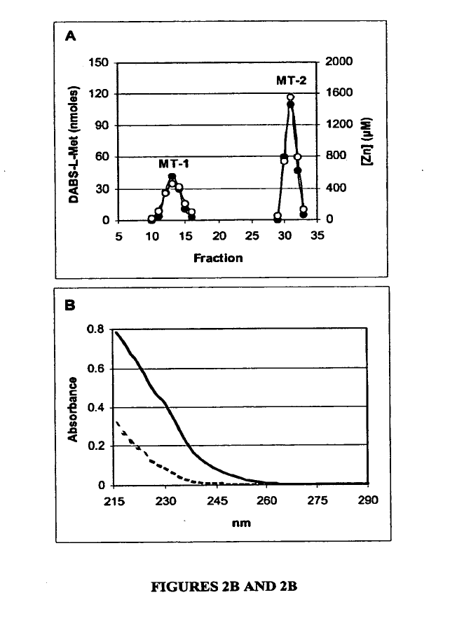

The heat stability and EDTA requirement suggested that the active factor might be a MT, and the heat-stable factor was further purified as described in Methods and elsewhere (Vasak, M. (1991) Methods Enzymol 205, 41-44). FIG. 2A shows the elution profile from a DE-52 cellulose column, the last step in the purification. Two distinct peaks of reducing activity were observed, and the fractions in both peaks were active in the Msr assay using hMsrB3 only in the presence of EDTA. The purification profile suggested that the two peaks correspond to MT-1 and MT-2 based on their elution from the DE-52 column.

Because of the requirement for EDTA for the fraction to be active with hMsrB3, metal analyses were initially performed on purified preparations using ICP-MS. Zinc was found in significant amounts (60,795 ppb) with trace levels of copper and silver (688 and 739 ppb, respectively). Besides using nanopure water, no special precautions were taken to remove trace metals so the source of these trace metals in the protein sample is unknown. As shown in FIG. 2A, the active, highly-purified fractions from the DE-52 column contained high levels of zinc that co-eluted with the fractions active in the Msr assay. The amount of MT could be determined spectrophotometrically (ε₂₂₀=48,600 M⁻¹ cm⁻¹ at pH 2.0), and zinc analyses using the PAR reagent (see methods) showed that there were close to 7 zinc atoms per mole of MT in each fraction. Although zinc appears to be the major metal associated with the active factor, we cannot eliminate the presence of lower levels of other metals in the sample. FIG. 2B shows the UV spectrum of a fraction from peak 2 from the DE-52 column (both peaks displayed similar spectral characteristics). It can be seen that the active factor has high absorption in the 200-250 nm range but essentially no absorbance at 280 nm, indicating the absence of aromatic amino acids. Upon acidification, the high UV absorption is markedly decreased. On SDS-PAGE, the purified protein, as well as a commercial rabbit liver MT preparation, migrated as a diffuse band in the 13-16 KDa range, double the size of Zn-MT, which is approximately 6 KDa. This could be due to the unique shape of the protein or the presence of dimers through intermolecular bond formation. The liver MT obtained from a commercial source also supported hMsrB3 activity in the presence of EDTA. The presence of zinc as well as the spectral and other characteristics of the active fractions indicated that the two peaks off the DE-52 column were Zn-MT-1 and Zn-MT-2. These peak fractions were further analyzed by electrospray mass spectrometry, and the molecular weights matched those of bovine MT-1 and MT-2 (5,987 and 6,013, respectively). EDTA removed >90% of the zinc from Zn-MT in less than 10 minutes, as measured by the appearance of free SH groups. It was concluded that the purified factor is a Zn-MT which, in the presence of EDTA, is converted to the metal-free reduced thionein (T), and that T, because of the high content of cysteine residues, is able to supply the reducing system for the Msr reaction. The results shown below used Zn-MT-2, although similar results were obtained with Zn-MT-1.

As seen in Table 2, the purified Zn-MT is not a specific reducing agent for hMsrB3 since it also supports eMsrA, eMsrB and bMsrA, dependent on EDTA. However, to our surprise, the liver factor showed very little activity with hMsrB2 under the conditions used in Table 2.

Msr proteins were incubated as described in Materials and Methods with either 20 nmoles of purified Zn-MT or with 15 mM DTT. Incubations with Zn-MT routinely contained 5 mM EDTA, and no significant activity was detected in the absence of EDTA. The amounts of proteins used were: eMsrA, 1.6 μg; eMsrB, 5.4 μg; bMsrA, 2.0 μg; hMsrB2, 2.7 μg; hMsrB3, 2.3 μg.

TABLE 2
Comparison of the activity of Msr proteins
in the presence of Zn-MT or DTT.

nmoles DABS-met

| Msr proteins | Zn-MT | DTT |
|---|---|---|
| eMsrA | 33.9 | 45.7 |
| eMsrB | 8.3 | 27.8 |
| bMsrA | 14.1 | 38.9 |
| hMsrB2 | 0.9 | 27.1 |
| hMsrB3 | 18.0 | 53.7 |

### Example 3

### T can Function in the Msr System in the Absence of EDTA

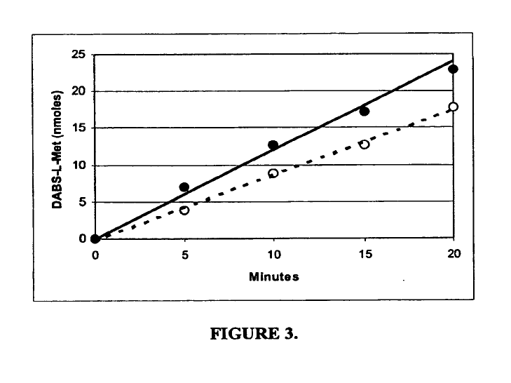

Although it appeared likely that the requirement for EDTA was to release zinc from Zn-MT to form T, it was important to demonstrate directly that T could serve as the reducing agent for the Msr system. T was prepared as described in Methods and tested with hMsrB3 as shown in FIG. 3. It can be seen that hMsrB3 activity was supported by both T and Zn-MT, although T was active in the absence of EDTA, whereas Zn-MT required EDTA for activity. Shorter incubations were used for these experiments to minimize the oxidation of T that occurred at neutral pH. T also was active with MsrA in the absence of EDTA. These results support the view that the requirement for EDTA with Zn-MT is to release the zinc from Zn-MT and form T, and that T is able to provide the reducing system for the Msr enzymes.

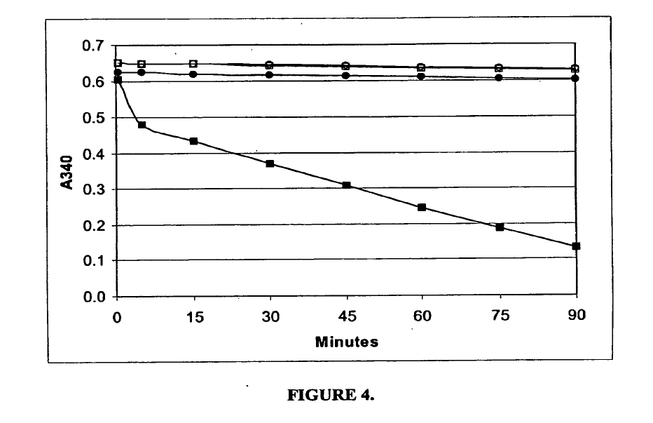

Trx can reduce T(o): The reaction mechanism for both MsrA and MsrB involves the formation of an oxidized enzyme intermediate that must be reduced for the Msr protein to act catalytically. If T is capable of reducing oxidized Msr, the T would become partly or fully oxidized to T(o), and ideally there should be an enzymatic system that could regenerate T and permit it to recycle. Partially oxidized T(o) was prepared as described in Methods. This material had generally lost about 50-60% of its free SH groups but still remained mostly soluble (see Methods). Any insoluble material that was formed was removed by centrifugation. Trx was considered a possible candidate to reduce T(o), and this could be shown directly by measuring NADPH oxidation in the presence of the Trx reducing system and T(o). As shown in FIG. 4, the oxidation of NADPH was dependent on Trx, Trx reductase and T(o). In addition, as shown in Table 3, T(o) could support hMsrB3 activity only in the presence of the complete Trx reducing system (line 1) but not in the absence of either Trx, Trx reductase or NADPH (lines 3-5). As discussed previously, the Trx system alone showed very low activity (line 2). It is also apparent from the results in Table 4 that the free SH groups remaining in the partially oxidized T(o) cannot support the Msr reaction, indicating that the SH groups in T are not all equivalent with respect to their ability to function as a reducing agent for the Msr system. The results in FIG. 4 and Table 3 indicate that disulfide bonds in T(o) can be reduced by the Trx system. Thus, Trx may be one of the cellular agents that can enable oxidized thionein to recycle and function as a metabolic reducing system.

In contrast to the results with hMsrB3, hMsrB2, which had low activity with either Trx or Zn-MT (see above, Table 2), was also not stimulated when both T(o) and the Trx reducing system were present.

Until the present studies, it has been assumed that Trx was the biological reducing system in cells for all of the Msr proteins. The initial experiments using eMsrA indicated that Trx was the biological reducing agent in agreement with earlier experiments showing the inability of Met(o) to support growth of an E. coli Trx/met double mutant (Weissbach, H., et al. (2002) Arch Biochem Biophys 397, 172-178; Brot, N. & Weissbach, H. (1983) Arch Biochem Biophys 223, 271-281). In those experiments, it was shown that Met(o) could support growth of a Met-requiring strain, but not if the organism was also Trx deficient, indicating that Trx is necessary for the conversion of free Met(o) to Met in E. coli. The present experiments are in agreement with these earlier results. It appears that MsrA from both bacterial and mammalian sources utilizes Trx very efficiently as does MsrB from E. coli. However, the studies reported here show that hMsrB2 and hMsrB3 (and presumably MsrB proteins from other mammalian sources) use Trx very poorly.

Recently, Kim and Gladyshev (Kim, H. Y. & Gladyshev, V. N. (2005) PLoS Biol 3) postulated that in MsrB1 a cysteine was required, in addition to selenocysteine, for Trx to function. In contrast, with MsrB2 and MsrB3, only the active-site cysteine was required, and Trx was thought to directly reduce the sulfenic acid intermediate on the enzyme. It seems clear, from the low activity using Trx with both MsrB2 and MsrB3, that this reaction is not efficient, which raises the possibility that Trx may not be the direct biological reducing system for MsrB2 and MsrB3. The ability of a heated bovine liver extract to support Msr activity with hMsrB3, in the absence of an exogenous reducing system, was the first evidence that animal cells contain a factor that, in the presence of EDTA, can substitute for Trx in this reaction. The identification of Zn-MT as the active factor was based on the heat stability, purification characteristics, absorption spectra at neutral and acidic pH, gel analysis, metal determination and molecular weight analyses. The role of EDTA appears to be to release the zinc from the Zn-MT to form T, the apoform of MT, which can function as a reducing agent because of its high content of cysteines. In support of this it was shown that T, prepared by acid treatment could function as a reducing agent in the Msr system without EDTA. It is known that T is a small protein having about 60 amino acids, with a molecular weight in the range of 6-7 KDa. Of the total amino acids, about one third are cysteines, which could make this protein an important cellular source of sulfhydryl groups. For many years it was felt that the MT's primary function was to scavenge free radicals and/or detoxify metals. However, in 1998, Maret and Vallee, postulated that the zinc-sulfur clusters in MT also acted as a sensor for the redox state of the cell (Maret, W. & Vallee, B. L. (1998) Proc Natl Acad Sci USA 95, 3478-3482). Oxidation of the Zn-MT resulted in release of the zinc so it could be mobilized within the cell, whereas under reducing conditions T would efficiently bind zinc. Thus, the major role of MT may be to control cellular zinc mobilization as a function of the redox state of the cell. However, there does not appear to be much information on other possible functions of T, the reduced apoform of MT, in addition to its critical role in binding zinc. Our results on the ability of T to supply the reducing system for some of the Msr proteins support this conclusion and link the MT proteins to another cellular antioxidant system.

Although the results indicate that T can supply the reducing system for all of the Msr enzymes tested, with the exception of hMsrB2, it is clear that the Trx system is the preferred reducing system for MsrA and eMsrB. If there is an important reducing role of T it is with hMsrB3. One of the unexplained and surprising findings in this study was the failure of T (or Zn-MT and EDTA) to stimulate hMsrB2. As mentioned, all of the other Msr proteins tested showed significant activity with Zn-MT in the presence of EDTA. Since MsrB2 and MsrB3 are both zinc proteins, they are thought to have similar reaction mechanisms (Kim, H. Y. & Gladyshev, V. N. (2004) Mol Biol Cell 15, 1055-1064; Kim, H. Y. & Gladyshev, V. N. (2005) PLoS Biol 3) which makes the lack of activity of T (the active agent) with hMsrB2 puzzling. One possibility is that different sulfhydryls on T react with hMsrB3 and hMsrB2. Thus, the active sulfhydryls in T that can interact with hMsrB3 cannot reduce the oxidized hMsrB2 intermediate.

Since both MT-1 and MT-2 gave similar results in supporting Msr activity in the presence of EDTA, we have assumed that T derived from other MTs, such as MT-3 (found in brain and reported to have growth inhibitory activity) and MT-4 would behave in a similar fashion. The electrospray mass spectrometry analysis did not show the presence of MT-3 in our samples. However, it is possible that slight structural differences in the MTs might be important, and it will be necessary to test the individual MT species for their ability to provide a reducing system for the Msr enzymes.

Yang et al (Yang, Y., Maret, W. & Vallee, B. L. (2001) Proc Natl Acad Sci U S A 98, 5556-5559) have reported that as much as 50% of the total MT in mammalian tissues is present as T. Thus, the high concentration of T in tissues is consistent with a possible role of T as a cellular reducing agent, especially if there are mechanisms to regenerate T from T(o), as shown here with Trx. The heat step should have destroyed any T in our liver preparations, although as shown in FIG. 1, there was a slight activity in the heated S-100 in the absence of EDTA that could have been due to T that was not destroyed by the heat step.

Since oxidative stress is believed to release zinc and other metals from MT, one can postulate a reaction sequence, summarized in FIG. 5, in which cells, under oxidative stress, mobilize zinc from Zn-MT for use for the hundreds of zinc-containing proteins. The loss of the zinc from MT as a result of oxidation would yield partly or fully oxidized T(o). As postulated in FIG. 5, T(o) can be reduced to −T by the Trx system, and evidence for this is shown above in Table 3 and FIG. 4. T can serve as a cellular reducing agent and reduce the oxidized Msr intermediates, either an enzyme-bound disulfide or sulfenic acid.

We have evidence that T can act catalytically, indicating that more than one cysteine on T is functional. Trx may be only one of the possible cellular reducing systems that can reduce T(o). It is known that oxidized glutathione can oxidize MT and cause the release of Zn from Zn-MT and that reduced glutathione can reduce T(o), which can bind zinc. Certain selenium compounds can accelerate these reactions which might also be a clue to what can occur in vivo. Further studies are required to determine whether the interaction between the Msr system and T has physiological relevance, especially since both may play an important role in protecting cells against oxidative damage. In addition, the possibility should be considered that T may be playing an important role as a cellular reductant for other systems.

TABLE 3
Thioredoxin stimulates the activity of MsrB3 in the presence of T(o).

| | | | | Trx | |
|---|---|---|---|---|---|
| # | T(o) | Trx | NADPH | reductase | nmoles DABS-met |
| 1 | + | + | + | + | 23.4 |
| 2 | − | + | + | + | 2.4 |
| 3 | + | − | + | + | 0.5 |
| 4 | + | + | − | + | 3.4 |
| 5 | + | + | + | − | 3.9 |

The incubations contained hMsrB3 (2.3 μg) as described in Methods. Where indicated, T(o), 8.3 nmoles, Trx, 10 μg, Trx reductase, 2.4 μg and NADPH, 100 nmoles were added. In this experiment, with the amount of hMsrB3 used, 52.7 nmoles DABS-met were formed in the presence of 15 mM DTT.

### Example 4

### Selenium Compounds can Stimulate the Methionine Sulfoxide Reductase Enzymes

The methionine sulfoxide reductases (Msr) are a family of enzymes that can reduce methionine sulfoxide (met(o)) in proteins and/or free met(o). The reduction of met(o) in proteins is catalyzed by either MsrA, which reduces the S epimer of met(o) (met-S-(o)), or MsrB, which reduces the R epimer of met(o) (met-R-(o)). Previous genetic studies with MsrA have shown that this enzyme plays an important role in protecting cells against oxidative damage and may also be involved in aging. Although thioredoxin (Trx) has been accepted as the reducing agent for MsrA, the above studies have shown that two of the members of the MsrB family, MsrB2 and MsrB3, do not use Trx efficiently. In a search for another reducing system for these MsrB enzymes, it was discovered that thionein (T), the reduced apoprotein of metallothionein (MT), could function as a reducing system for MsrB3 and that Trx could reduce oxidized thionein (T(o)), permitting T to recycle.

Oxidation and reduction of Zn-MT, shows that selenium compounds, such as selenocystamine (SeC), can markedly increase the release or uptake of zinc by MT, dependent on the oxidation state of the protein. As an example, SeC has been shown to oxidize Zn-MT resulting in the release of zinc and the formation of T(o). In the presence of a reducing agent such as GSH, the SeC is reduced to selenocysteamine (SeCe), which markedly accelerates the reduction of the T(6) and the uptake of zinc to form Zn-MT. Trx reductase can reduce selenium compounds directly (in the absence of Trx) and the reduced selenium compounds can function as reducing agents for a variety of enzymatic systems. One of the members of the Msr family, MsrB1, is a selenoprotein, and mice on a selenium-deficient diet have lower levels of MsrB activity, presumably due to reduced activity of MsrB1. In the present studies we have examined the effect of SeC on the activity of several Msr enzymes in the presence of Trx and T.

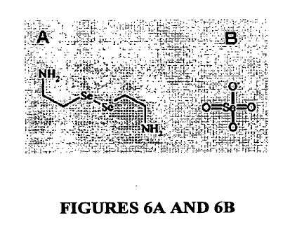

Results: In looking for agents that could stimulate the reaction of the Msr enzymes with either Trx or T, the selenium compound SeC was tested because of its ability to accelerate oxidation reduction reactions involving MT. (FIGS. 6A and 6B shows the structure of SeC, FIG. 6A, and selenite, FIG. 6B). Table 4 summarizes the results using 5 members of the Msr family, eMsrA, eMsrB, bMsrA, hMsrB2 and hMsrB3. The activity was measured in the presence or absence of SeC using either the complete Trx system (NADPH, Trx reductase and Trx) or the Trx system lacking Trx, to determine whether hydrogen transfer to the Msr enzyme(s) required Trx. SeC by itself had no activity with any of the Msr enzymes. As seen in Table 4, using the complete Trx system (columns 1 and 2), SeC had only a small effect on the activity of the E. coli enzymes, eMsrA and eMsrB (10-20% stimulation). These enzymes have been known to use the Trx system efficiently. In contrast, the activity of the bovine MsrA (bMsrA) and the two human MsrB's (hMsrB2 and hMsrB3) are markedly stimulated by the presence of SeC. The bMsrA activity increases more than 3-fold, and the activity of both hMsrB2 and hMsrB3 increase 50-100 fold in the presence of SeC. Previously we have shown that the Trx system is a very poor reducing system for hMsrB2 and hMsrB3, and this is also seen in Table 4 (column 1). In fact, until now the only reducing agent that has given good activity with hMsrB2 has been DTT. However, it is evident from the results in Table 4 that the Trx system can function with hMsrB2 in the presence of a selenol compound. Table 4, columns 3 and 4 show the results of similar experiments in which Trx has been omitted from the Trx reducing system. In column 3 it is seen that Trx reductase and NADPH, in the absence of Trx, give very low activity with all of the Msr enzymes tested. However, in the presence of SeC, all of the enzymes show significant activity, indicating that Trx reductase can transfer hydrogen from NADPH to SeC to form SeCe and that SeCe can supply the reducing power for the Msr enzymes.

Table 5 shows the results of similar experiments with T. As shown above, T can support the reaction with all of the Msr enzymes tested with the exception of hMsrB2. However, in the presence of SeC there is also a marked stimulation of the activity with all of the Msr enzymes including hMsrB2. Once again the most striking effects are seen with hMsrB2, hMsrB3 and bMsrA. These results indicate that T can also reduce SeC, although not as efficiently as the Trx system, and that the SeCe formed can again supply the reducing system for all of the Msr enzymes.

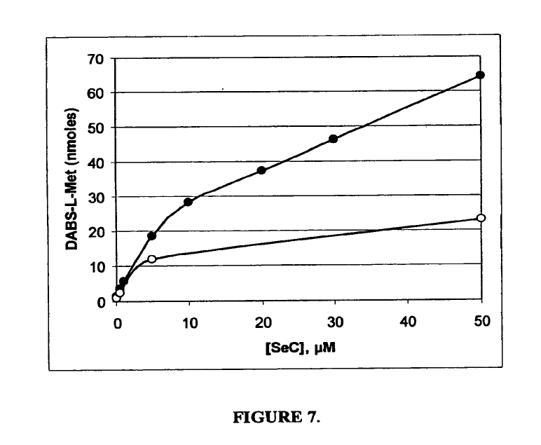

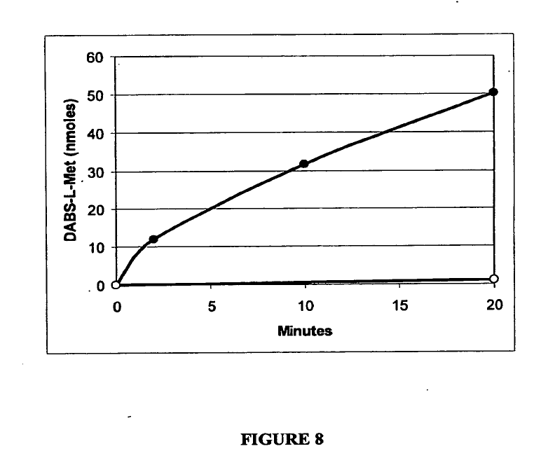

FIG. 7 shows the effect of SeC concentration on the activity of hMsrB3 using either NADPH and Trx reductase or T as the primary reducing agents. Under the assay conditions a maximal stimulation is seen at a final concentration of 50 μM SeC. FIG. 8 shows a time curve for hMsrB3 activity using the Trx reducing system (NADPH and Trx reductase) in the presence or absence of 50 μM SeC. The reaction is linear for up to 60 minutes and the marked stimulation by SeC is apparent at all time points.

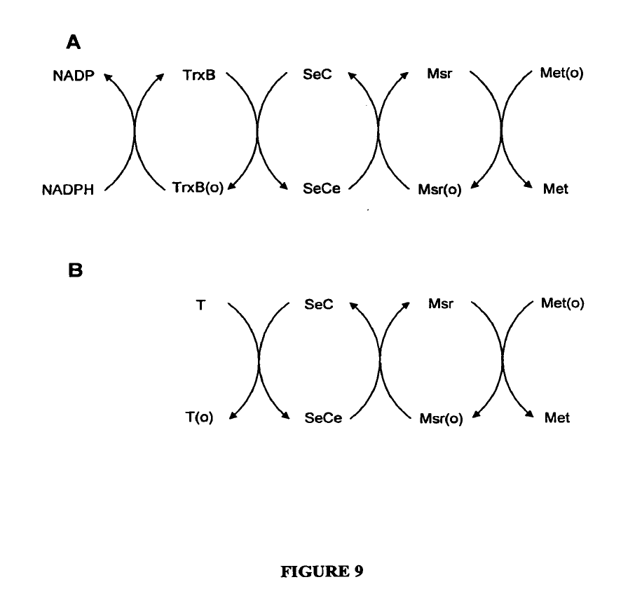

It appears that the Trx system is the primary reducing system for the Msr enzymes as well as eMsrB. However, the low activity of both hMsrB2 and MsrB3 with Trx as the reducing system suggested that there may be another reducing system in mammalian cells for these enzymes. The recent finding that T, the reduced apoform of metallothionein, can reduce hMsrB3 and that oxidized thionein (T(o)) could be reduced by the Trx system has focused attention on the possible role of T as a cellular reducing agent, in addition to its role in binding metals. Since neither Trx nor thionein functioned with hMsrB2 it seemed clear that there may be other factors that played a role in supplying the reducing system for hMsrB2 and, very likely, hMsrB3. It has been known that selenium compounds can accelerate the binding and release of zinc from Zn-MT in the presence of GSH and GSSG, respectively. In addition previous work has demonstrated that Trx reductase can reduce oxidized selenium compounds such as SeC. The present studies confirm that SeC can be reduced by NADPH and Trx reductase and also by T. Once reduced the SeCe formed is a potent reducing agent for hMsrB2 and hMsrB3. Although Trx was a poor reducing agent for both hMsrB2 and hMsrB3, it is a very effective reductant for the MsrB enzymes in the presence of SeCe, which appears to be an intermediate hydrogen carrier between the Trx and the oxidized MsrB enzyme intermediate. A similar situation appears true for T, which also is a much more efficient reducing agent for the MsrB enzymes in the presence of a SeC. These reactions are summarized in FIG. 9. In contrast, the ability of SeC to stimulate the Trx dependent reaction with eMsrA and eMsrB was much less. In general, with the E. coli enzymes, there was less than a twofold stimulation above that seen with the Trx system alone, whereas with the MsrB enzymes the SeC stimulated the reaction with Trx more than 50 fold. The identification of a naturally occurring compound, perhaps a selenium derivative, in mammalian cells that can act as an intermediate hydrogen carrier between Trx (or T) and the MsrB enzymes, similar to what SeC is doing in the in vitro studies described here is a further goal of these studies.

Examples of selenium derivatives include, but not limited to: selenium dioxide; selenium sulfide; sodium selenite; sodium selenate; zinc selenite; copper selenate; barium selenite; ferrous selenide; hydrogen selenide; seleneous acid; selenic acid; sodium selenide; diphenyl selenide; benzeneseleninic anhydride; benzeneseleninic acid; diphenyl diselenide; selenophenol (phenylselenol); selenium aspartate; phenylselenenyl chloride; phenylselenenyl bromide; selenourea; L(+) selenomethionine; selenium tetrabromide.

TABLE 4

| | | Complete | Trx | Trx |
|---|---|---|---|---|
| | Complete | Trx system + | reductase + | reductase + |
| Enzyme | Trx system | SeC | NADPH | NADPH + Sec |
| hMsrB3 | 1.1 | 52.9 | 0.9 | 48.9 |
| eMsrA | 15.3 | 18.6 | 1.9 | 12.2 |
| bMsrA | 13.1 | 45.9 | 0.6 | 41.2 |
| eMsrB | 22.5 | 24.2 | 0 | 5.6 |
| hMsrB2 | 0.3 | 29.2 | 0 | 9.1 |

TABLE 5

| Enzyme | T | T + SeC |
|---|---|---|
| hMsrBS | 1.3 | 17.7 |
| eMsrA | 3.6 | 5.7 |
| bMsrA | 3.3 | 14.7 |
| eMsrB | 0.4 | 3.2 |
| hMsrB2 | 0 | 4.6 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A pharmaceutical composition comprising an isolated biological agent wherein the isolated biological agent is a thionein (T).

2. The pharmaceutical composition of claim 1, wherein the thionein is a zinc metallothionein.

3. The pharmaceutical composition of claim 1, wherein the composition further comprises compounds that protect against oxidative damage.

4. The pharmaceutical composition of claim 1, wherein the composition further comprises selenium and selenium derivatives.

5. The pharmaceutical composition of claim 4, wherein the selenium derivatives comprise at least one of: selenocystamine, selenocysteamine; selenium dioxide; selenium sulfide; sodium selenite; sodium selenate; zinc selenite; copper selenate; barium selenite; ferrous selenide; hydrogen selenide; seleneous acid; selenic acid; sodium selenide; diphenyl selenide; benzeneseleninic anhydride; benzeneseleninic acid; diphenyl diselenide; selenophenol (phenylselenol); selenium aspartate; phenylselenenyl chloride; phenylselenenyl bromide; selenourea; L(+) selenomethionine; and, selenium tetrabromide.

6. The pharmaceutical composition of claim 1, wherein the composition comprises ethylenediaminetetraacetic acid (EDTA).

7. A method of reducing, preventing or reversing oxidative damage in a cell, the method comprising the steps of:
(a) providing a pharmaceutical composition comprising an isolated biological agent wherein the isolated biological agent is a thionein (T), the compound being a substrate for at least one Msr enzyme;
(b) providing a cell expressing at least one Msr enzyme, said cell comprising or being exposed to reactive oxygen species; and
(c) contacting the cell with an amount of the compound sufficient to reduce, prevent, or reverse oxidative damage in the cell by said reactive oxygen species.

8. The method of claim 7, wherein the cell is within an animal subject.

9. The method of claim 7, wherein the animal subject has a condition or disorder associated with oxidative damage.

10. The method of claim 7, wherein said method further comprises administering to said cell a pharmaceutical composition comprising sulindac, or sulindac metabolites, or sulindac derivatives or combinations thereof.

11. The method of claim 7, wherein the selenium derivatives comprise at least one of: selenocystamine, selenocysteamine; selenium dioxide; selenium sulfide; sodium selenite; sodium selenate; zinc selenite; copper selenate; barium selenite; ferrous selenide; hydrogen selenide; seleneous acid; selenic acid; sodium selenide; diphenyl selenide; benzeneseleninic anhydride; benzeneseleninic acid; diphenyl diselenide; selenophenol (phenylselenol); selenium aspartate; phenylselenenyl chloride; phenylselenenyl bromide; selenourea; L(+) selenomethionine; and, selenium tetrabromide.

12. A method of treating a patient suffering from a condition or disorder associated with oxidative damage, the method comprising the steps of:
(a) providing a pharmaceutical composition comprising an isolated biological agent wherein the isolated biological agent is a thionein (T), the compound being a substrate for at least one Msr enzyme;
(b) administering to a patient the pharmaceutical composition; and,
(c) contacting a cell with an amount of the compound sufficient to reduce, prevent, or reverse oxidative damage in the cell by said reactive oxygen species; and,
treating a patient suffering from a condition or disorder associated with oxidative damage.

13. The method of claim 12, wherein said pharmaceutical composition further comprises selenium and/or selenium derivatives.

14. The method of claim 12, wherein the selenium derivatives comprise at least one of: selenocystamine, selenocysteamine; selenium dioxide; selenium sulfide;
sodium selenite; sodium selenate; zinc selenite; copper selenate; barium selenite;
ferrous selenide; hydrogen selenide; seleneous acid; selenic acid; sodium selenide;
diphenyl selenide; benzeneseleninic anhydride; benzeneseleninic acid; diphenyl diselenide; selenophenol (phenylselenol); selenium aspartate; phenylselenenyl chloride; phenylselenenyl bromide; selenourea; L(+) selenomethionine; and, selenium tetrabromide.

15. A method of inducing metallothionein production in a cell comprising:
(a) providing a pharmaceutical composition comprising an isolated biological agent wherein the isolated biological agent is a thionein (T), the compound being a substrate for at least one Msr enzyme;
(b) providing a cell expressing at least one Msr enzyme, said cell comprising or being exposed to reactive oxygen species; and
(c) contacting the cell with an amount of the compound sufficient to induce metallothionein production.

16. The method of claim 15, wherein the cell is within an animal subject or in vitro.

17. The method of claim 15, wherein said method further comprises administering to said cell a pharmaceutical composition comprising sulindac, or sulindac metabolites, or sulindac derivatives or combinations thereof.

18. The method of claim 15, wherein the selenium derivatives comprise at least one of: selenocystamine, selenocysteamine; selenium dioxide; selenium sulfide; sodium selenite; sodium selenate; zinc selenite; copper selenate; barium selenite; ferrous selenide; hydrogen selenide; seleneous acid; selenic acid; sodium selenide; diphenyl selenide; benzeneseleninic anhydride; benzeneseleninic acid; diphenyl diselenide; selenophenol (phenylselenol); selenium aspartate; phenylselenenyl chloride; phenylselenenyl bromide; selenourea; L(+) selenomethionine; and, selenium tetrabromide.

19. The method of claim 15, wherein the composition comprises ethylenediaminetetraacetic acid (EDTA).

20. A method of diagnosing a patient suffering from a condition or disorder associated with oxidative damage comprising:
obtaining a biological sample from the patient;
measuring metallothionein concentration in the sample; and,
diagnosing a patient suffering from a condition or disorder associated with oxidative damage.

21. The method of claim 20, wherein the patient is an animal.

22. The method of claim 20, wherein the biological sample is a cell, tissue, organ, blood or other fluids.
